(19) United States
(12) Patent Application Publication
(10) Pub. No.: US 2016/0263375 A1
(43) Pub. Date: Sep. 15, 2016

(54) WEARABLE DEVICE WITH WIRELESS BLUETOOTH MASSAGE FUNCTION

(71) Applicant: Ming-Lai WU, New Taipei City (TW)

(72) Inventor: Ming-Lai WU, New Taipei City (TW)

(21) Appl. No.: 14/658,025

(22) Filed: Mar. 13, 2015

(51) Int. Cl. A61N 1/32; H04W 4/02; H04W 4/00

(52) U.S. Cl. CPC A61N 1/322; H04W 4/008; H04W 4/02

(57) ABSTRACT

A wearable device with wireless Bluetooth massage function, which is mainly installed inside shoes, and which combines with the mobile device and APP to form interactive therapeutic massage shoes; said wearable device includes: an insole, with its surface defined with anode and cathode interfaces of conductive media, an electronic structure, having a motherboard, a Bluetooth chipset module, and a battery, and electrically connected to the insole; based on this, when the user is wearing the insole, the body electricity and the anode and cathode conductive media interfaces on the surface of the insole will form a complete electric cycle, and by manipulating the mobile device, options can be made to: in the electro-therapeutic massage mode, provide regular low-frequency electro-therapeutic massage; or in the physiological signal detection mode, acquire physiological feedback signals in real time and transmit them to the mobile device through Bluetooth for real-time judgment by the user, or to the cloud data safe for analysis and advice by experts.

### BACKGROUND OF INVENTION

1. Field of the Invention

The present invention relates generally to a medical device, and more particularly to a conductive sensor installed inside a shoe, which, when contacting with the human body, can form a complete electrical cycle with the human body electricity, and which can be operated by a mobile device through Bluetooth wireless transmission, so as to realize a wearable device with the function of low-frequency electrotherapy and massage, or even the function to detect and collect physiological information.

2. Description of Related Art

Nowadays, many people are leading a busy life. Most students and office workers do not have time for sport, and many are suffering from diseases of modern civilization. Particularly for most office workers who sit or stand for too long time every day, there are more chances of leg diseases. Plus, due to irregular diet and unbalanced nutrition, such diseases like obesity and diabetes are occurring to people of younger ages year after year.

Secondly, the situation of fewer children is becoming more and more serious. As a result, the number of senior people who need care is almost larger than that of young people. Due to oldness and functional degradation of some organs, chronic poor blood circulation is very common among seniors, plus nervous lesions and muscle atrophy in the legs are causing less and less walking ability. Some seniors are even suffering from diabetes and are lying in the bed all day and all night. Without turning the body and patting on the back, the situation of poor blood circulation will become worse; moreover, nowadays there are more and more senior people who are living alone. Their children are working in other counties or cities and are unable to visit and look after them frequently. This phenomenon is not only causing social problems and higher medical expenses, but also adding heavy medical burdens to families with elderly people needing care. Such a situation may happen to anybody. Once it happens, it is obviously a great blow.

Although there are developments of medical and health apparatus to cope with the above situation, most devices are of large scale and sophisticated design, and are provided at specific medical sites. The limited application and high cost can hardly benefit the general public.

Massage and rehabilitation devices sold in the market may help solve the problems of poor circulation and metabolism, and enhance muscle strength in the legs, but such devices are usually fixed installations that are placed in medical sites or at home and can only work with electric power. For patients requiring frequent therapy, it is very inconvenient if they do not have the device at home; Moreover, during the therapy, the patient must sit on the device or installation and may not move freely or do something else. Therefore, it is not so flexible.

Due to new technologies, there are new developments of wearable 3C products, such as smart watches, smart bracelets etc. Their main function is to detect physiological information of the human body, including heart beat, respiration, skin surface temperature, movement speed etc. Based on this, during the wearing, the physiological situation of the wearer can be monitored in real time; however, such products must contact with the human body, and usually, due to grease or sweat on the skin of the wearer, incomplete contact may occur and may affect the accuracy of the detected physiological information. If using full-contact styles, such as stickers, long-term adhesion may cause skin allergy or rash to the wearer; the most annoying thing is that, such electronic products can not be thoroughly washed. They can only be cleaned partially and are not very durable and failure prone.

In addition, some medical organizations are currently offering so-called remote caring systems. A medical caring platform is built inside the medical organization, and elderly people or critically ill patients needing care wear detecting devices with multiple sensors. Through networking, the physiological information of the person under care is monitored in real time. In this way, the medical organizations are able to offer convenient, accurate and efficient medical information services; however, the detecting devices used in such systems also have difficulty in wearing. They are hard to wear without assistance of medical professionals. Incorrect wearing may lead to inaccuracy of the detected and collected physiological information, and wearing the devices will become meaningless.

Therefore, it is the basic spirit of the present invention to develop a product that can be incorporated into daily life, with a simple, easy and user-friendly interface, with no need for assistance by any professional person. The product will further offer functions of low-frequency electro-therapeutic massage and remote medical care. Meanwhile, it can monitor personal health in real time, so as to reduce dangers when going out. The product will offer such flexibility that the user can easily use either at home or when going out. Also, the product can be thoroughly washed for repeated usage and will have a long lifecycle.

### SUMMARY OF THE INVENTION

The main objective of the present invention is to provide a wearable device with wireless Bluetooth massage function, which, based on an ergonomic and user-friendly design, can be placed inside shoes so the users can use in daily life without changing their shoe-wearing habits. The wearable device is a conductive sensor, which, when contacting with the human body, can form a complete electrical circle with the human body electricity, and which can be operated by a mobile device through Bluetooth wireless transmission, to provide a function of low-frequency electrotherapy and massage for the user wearing the shoes, and to collect the physiological information of the user. In this way, it can offer massage and healthcare anytime and anywhere and meanwhile can detect the personal physiological information and location of the user in real time, and report emergencies to reduce the safety of the user when going out.

The second objective of the present invention is to provide a wearable device with wireless Bluetooth massage function, which, in view of the fact that, as a habit, most users who wear shoes will also wear socks, designed with accompanying conductive socks, so that the electrical cycle between the human body and the conductive sensor will not be interrupted by ordinary non-conductive socks, and that the wearable device can have an effective function of low-frequency electro-therapeutic massage or detecting and collecting the physiological information of the user.

Another objective of the present invention is to provide a wearable device with wireless Bluetooth massage function, which is accompanied by a control device, being the currently most convenient mobile devices that can be conveniently taken out, to select functions or designate one or more than one user through an APP, so that multiple seniors or ill patients needing care can be monitored at the same time, and that complete, accurate, efficient and timely medical rescue can be realized.

Another objective of the present invention is to provide a wearable device with wireless Bluetooth massage function, which incorporates a positioning chipset of the Global Positioning System with the wearable device, so that the location of the user can be monitored in real time through the mobile device, and that the personal safety of the user can be ensured.

A further objective of the present invention is to provide a wearable device with wireless Bluetooth massage function, which features good wear resistance, high strength, and long lifecycle, and which can be washed for repeated usages.

To realize the above objectives, the inventor of the present invention has designed a wearable device with wireless Bluetooth massage function, which incorporates the electronic structure of a conductive sensor built in shoes with a mobile device having a customized APP to form interactive massage shoes having the function of massage, health protection and remote care. Said wearable device includes: an insole, said insole partitioned into anode and cathode conductive media interfaces on its surface, and extended with anode and cathode connectors respectively on either end; and an electronic structure, which is electrically connected to the anode and cathode connectors, and which includes a motherboard and a Bluetooth chipset module to offer functions of low-frequency electro-therapeutic massage and detecting the human body physiological information.

Said wearable device further includes a mobile device, said mobile device having a built-in APP, a Bluetooth unit, and a display unit. Said mobile device can pair with the Bluetooth control chipset module of the insole through the Bluetooth unit, so as to issue commands for an electro-therapeutic massage mode or a physiological signal detection mode of the insole by manipulating the APP.

Information shown on the screen of the display unit of the said mobile device at least includes: variously selectable diagrams of physiological signals (e.g., heat beat, respiration, blood pressure, body temperature, body fat etc). When operation is made to select any physiological signal diagram, the collected physiological information will be presented by various statistical diagrams (e.g., bar chart, pie chart, or line chart) in the schema area, or by data in the data area.

Said shoes include slippers and house shoes for indoor applications, as well as leather shoes, sport shoes, leisure shoes and hiking shoes for outdoor applications.

Said battery can be button cell or any other power supply that is light, thin and small.

The electronic structure of the said wearable device can be further configured with a GPS positioning chipset.

The efficacies realized by the present invention reside in that: when the user is wearing the insole, the body electricity and the anode and cathode conductive media interfaces on the surface of the insole will form a complete electric cycle, and commands can be selectively issued to the insole through the APP of the mobile device, so that: (1) in the electro-therapeutic massage mode, the conductive media interfaces provides regular and cyclical low-frequency electro-therapeutic massage; (2) in the physiological signal detection mode, physiological feedback signals can be acquired through the conductive media interfaces, and transmitted to the smart phone through wireless Bluetooth, so that the user can make a judgment in real time, or transmitted to a database for expert advices.

For esteemed auditors to have a thorough understanding of the features, technical means and accomplished efficacies of the present invention, a preferred embodiment is taken as an example and detailed descriptions are provided below in accompany with drawings and reference numerals:

### BRIEF DESCRIPTION OF THE DRAWINGS

FIG. 1 is an embodiment view (1) of the wearable device of the present invention.

FIG. 2 is an embodiment view (2) of the wearable device of the present invention.

FIG. 3 is a structural view of the wearable device of the present invention.

FIG. 4 is a schematic view depicting the pairing between the electronic device and the mobile device of the wearable device of the present invention.

FIG. 5 is a schematic view of the operation screen of the mobile device of the wearable device of the present invention.

FIG. 6 is a schematic view of the schema area and data area of the mobile device of the wearable device of the present invention.

FIG. 7 is a schematic view of multiple Bluetooth pairings between the wearable device and the mobile device of the present invention.

FIG. 8 is a schematic view of the positioning of the wearable device and mobile device of the present invention.

FIG. 9 is another embodiment view of the wearable device of the present invention.

### DETAILED DESCRIPTION OF THE INVENTION

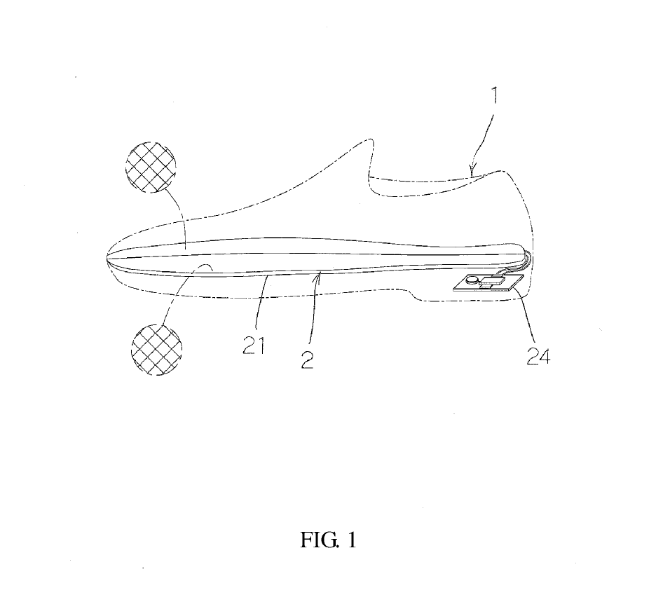

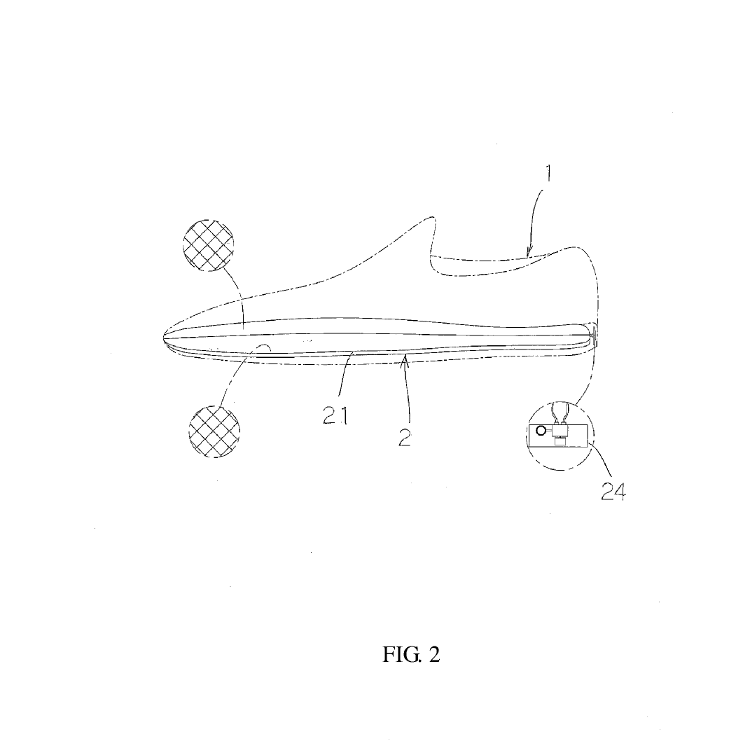

Referring to FIGS. 1 and 2, which disclose a wearable device with wireless Bluetooth massage function, said wearable device (2) being configured inside shoes (1), said shoes (1) can be various styles of shoes, such as leather shoes, slippers, sport shoes, leisure shoes, hiking shoes etc, which can be worn by ordinary users in daily life for optimal, ergonomic and user-friendly usages; Said wearable device (2) is mainly a conductive sensor, with its components including: an insole (21), which can be placed inside shoes (1), and an electrically connected electronic structure (24), which can be buried into the insoles of the shoes (1), or configured inside the shoes at the position facing the heel (as long as it does not affect wearing); furthermore, said electronic structure (24) can have an enclosure, to avoid damage to the inside.

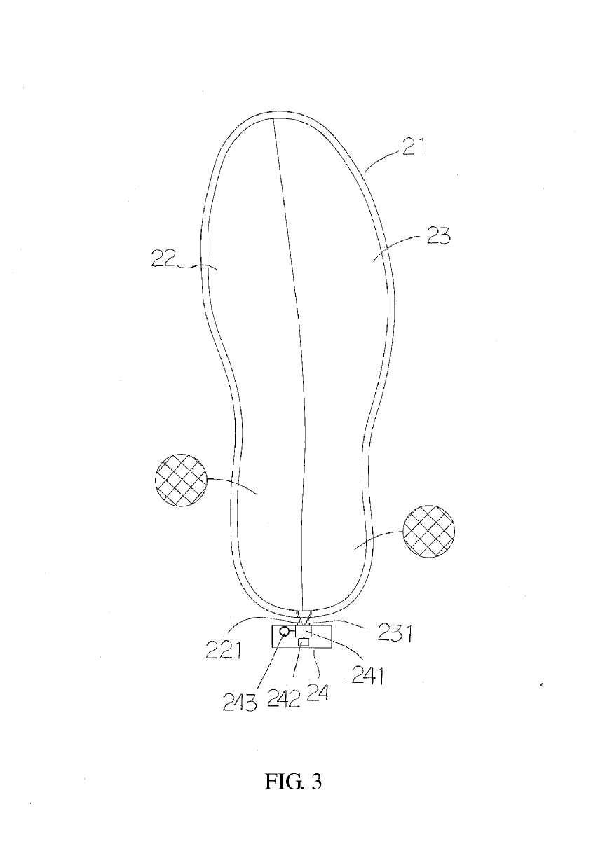

Referring to FIG. 3, the surface area of the insole (21) is partitioned and defined into an anode interface of conductive media (22) and a cathode interface of conductive media (23) respectively representing anode and cathode. There are no limitations to the allocated areas. The simplest example is to divide by the center line, making one side as the anode interface of conductive media (22), and the other side as the cathode interface of conductive media (23). It is necessary that the user's foot must contact with the anode and cathode interface of conductive media (22, 23) at the same time upon stepping. Meanwhile, either end of the anode/cathode interface of conductive media (22, 23) is respectively extended with an anode/cathode connector (221, 231).

Said electronic structure (24) is electrically connected to the anode and cathode connectors (221, 231), and at least includes: a motherboard (241), being the central control unit, a Bluetooth chipset module (242), being the media for signal transmission and reception, a battery (243), being the power source; based on the electrical conduction between the insole (21) and the foot surface of the human body, functions of low-frequency electro-therapeutic massage and human body physiological information detection can be realized.

In implementation, the surface of the insole (21) is covered by a metal mesh fabric to form the anode interface of conductive media (22) and cathode interface of conductive media (23), and the anode interface of conductive media (22) and the cathode interface of conductive media (23) are not electrically connected; said metal mesh fabric is knitted by superfine metal yarns, in the form of natural fibers wound and wrapped by superfine metal wires. In construction, superfine metal wires are finely ground out with abrasives (it is required that the metal wires must be superfine and will not break), and then coupled with natural fibers (including natural and artificial fibers). The thread diameter is 0.028-0.040 mm. The superfine metal yarn made in this way offers far higher strength than ordinary cotton yarns, ensuring significantly enhanced wear resistance and extended lifecycle of finished products. Meanwhile the adoption of natural fibers is to maintain softness and comfort. The metal fabric with the above construction combines good conductivity and softness and comfort.

The motherboard (241) inside the electronic structure (24) is preinstalled with a control IC with functions to control low frequency discharge and human body physiological information collection. Based on this, it can provide a function of low-frequency electro-therapeutic massage, and can transmit the collected human body physiological information to the mobile device (3) or the cloud data safe through the Bluetooth chipset module (242), so as to realize remote monitoring, remote medical care as well as emergency report.

The battery (243) inside the electronic structure (24) is mainly implemented with a button cell, but there is no limitation. Any independent power source that is light, thin and small can be used as the power supply.

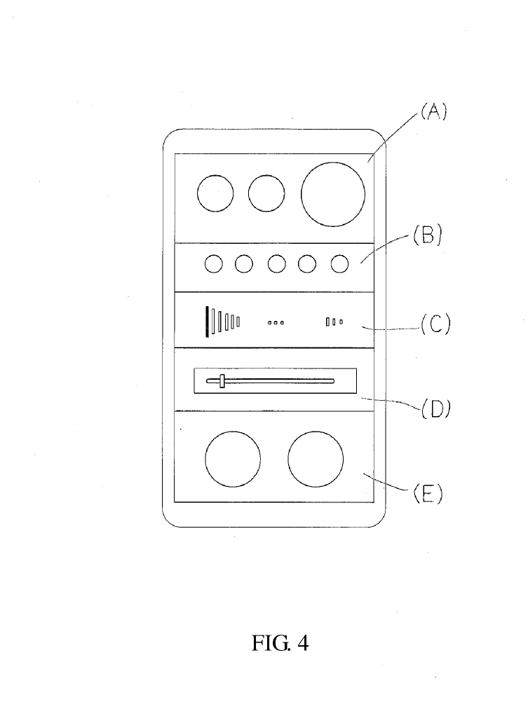

Referring to FIG. 4, which discloses a schematic view of the mobile device to couple with the electronic structure of the present invention. The mobile device (3) can be a smart phone or a tablet computer, having at least one APP (31), a Bluetooth unit (32), and a display unit (33). The mobile device (3) uses the Bluetooth unit (32) to pair and connect with the Bluetooth chipset module (242) inside the electronic structure (24) of the wearable device (2). Once connected, the APP (31) can be operated to issue a command to the wearable device (2) for a low-frequency electro-therapeutic massage, or for detection and collection of physiological signals through the electrical connection of the insole (21). The collected physiological information is then displayed on the display unit (33) of the mobile device (3) in graphic or data forms.

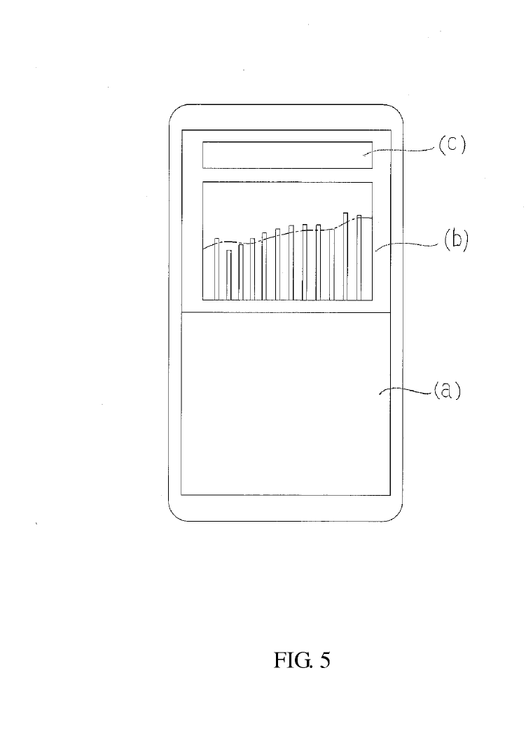

Referring to FIG. 5, to activate the massage function, the APP (31) can be operated through the display unit (33) to set the user and functions. The display at least includes: in case of single user: one foot (left and right)/both feet selection schema area (A), massage functions selection schema area (B), strength adjustment schema area (C), duration setting schema area (D), ON/OFF selection schema area (E), even music play can be designed so that, when the user is listening to the song, the rhythm can match the massage frequency to offer a more relaxing massage effect; in case of multiple users, a user settings selection schema area can be added, with the other functions being similar.

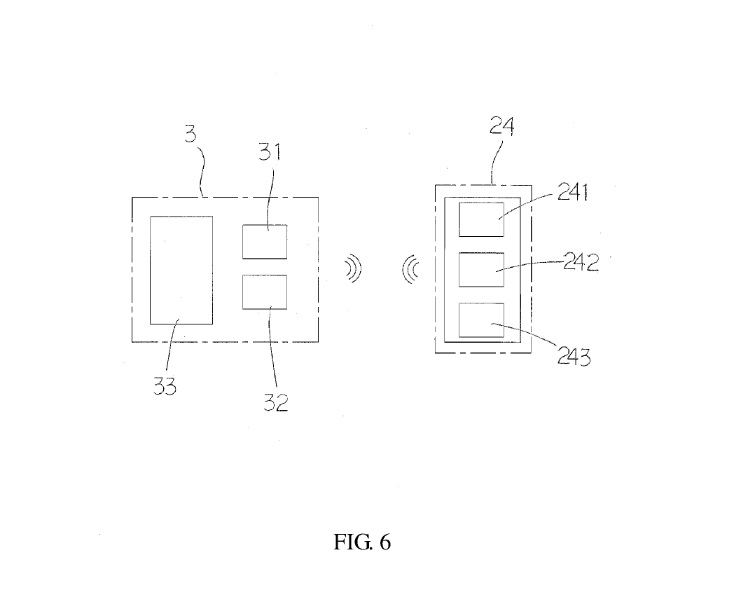

Referring to FIG. 6, when the user wants to operate the APP (31) to collect physiological information of the user, the screen of the display unit (33) will at least show: a selection schema area (a) for various physiological signals (such as heart beat, respiration, blood pressure, body temperature, body fat etc). When any of the physiological signal schemata is selected, the collected physiological information under this schema will be displayed in the schema area (b) in form of various statistical diagrams (such as bar chart, pie chart, line chart etc), or in the data area (c) in form of data.

Based on the above, in application, when the user's foot contacts with the anode and cathode interface of conductive media (22, 23) on the surface of the insole (21) of the wearable device (2), it will form an electrical cycle with the bioelectricity of the human body itself; at this time, a program command can be issued to the insole (21) through the APP (31) of the mobile device (3) for activities including: in the low-frequency electro-therapeutic massage mode, the anode and cathode interfaces of conductive media (22, 23) can offer regular low-frequency electro-therapeutic massage; in the physiological signal detection mode, based on the direct contact between the anode and cathode interfaces of conductive media (22, 23) and the foot skin, physiological information can be acquired in real time, and can further be transmitted to and displayed on the mobile device by means of wireless Bluetooth transmission, for real-time analysis and judgment by the user, or transmitted to the cloud data safe, for analysis or advice by medical experts for the purpose of remote care.

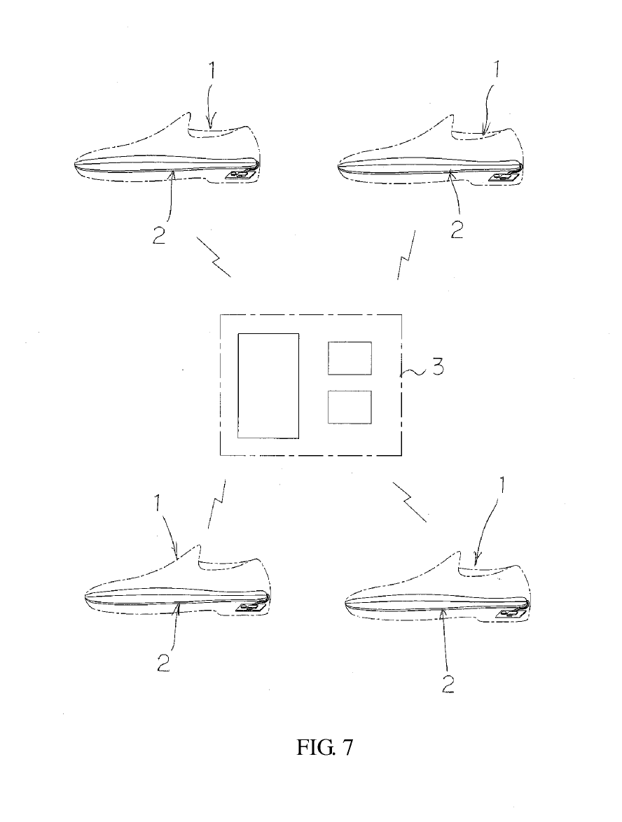

Referring to FIG. 7, in the implementation of the wearable device of the present invention, apart from one-to-one pairing, the mobile device (3) can also realize one-to-many pairing with multiple persons who wear shoes (1) with installation of the wearable device (2), and can shift between multiple connected users through the APP (31). And by shifting between the different user pages, the detected and collected physiological information can be respectively displayed in the schema area (b) or the physiological data area (c), for direct observation of the information data record, and for easy judgment of the user's physiological condition, or, can also be transmitted to the cloud data safe by means of wireless Bluetooth transmission, for further analysis, judgment and treatment of the information by medical experts.

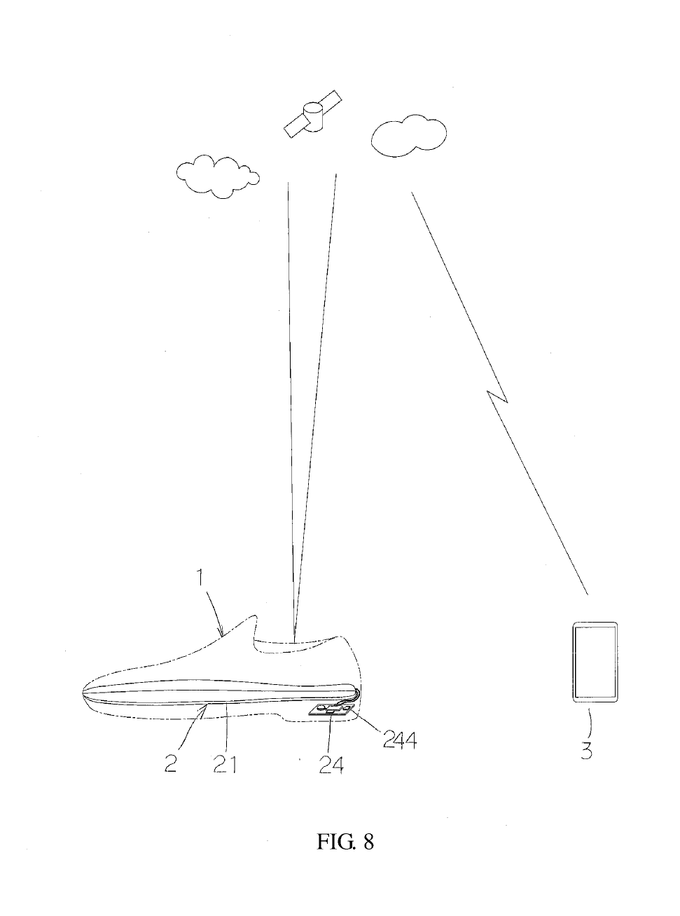

Referring to FIG. 8, the electronic structure (24) can further have a configuration of a GPS positioning chipset (244). Through signal transmission by the Bluetooth chipset module (242) and with the smart phone (3) and APP (31), the user wearing shoes (1) installed with the wearable device (2) can be precisely positioned, so that the travel of the user can be tracked and risk of the user's personal health and safety can be reduced.

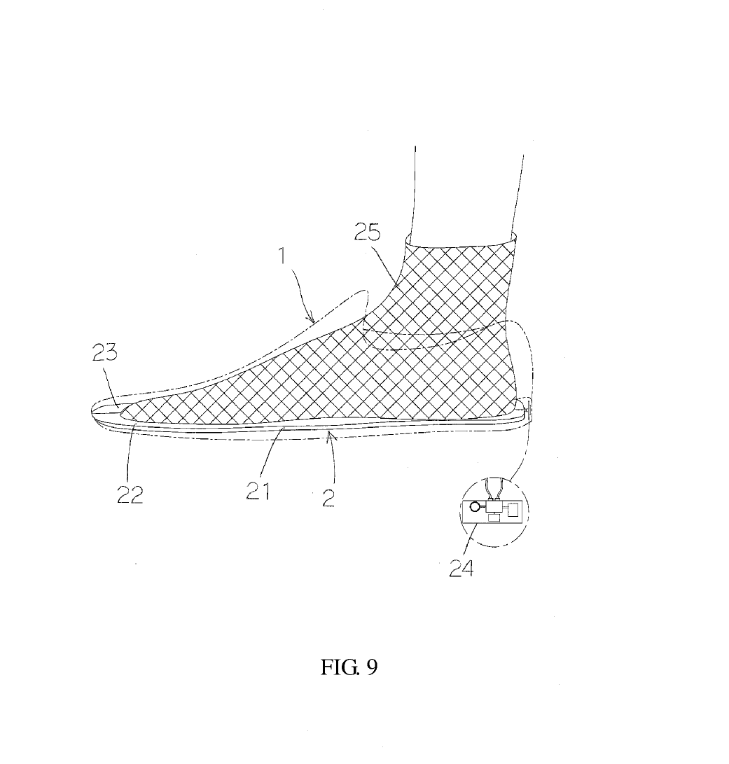

In application of the present invention, when the user wears the shoes (1), his/her feet can direct contact with the insole (21) of the wearable device (2) for electric conduction to realize the required low-frequency electro-therapeutic massage and detection of the human body physiological information. However, most users will usually wear socks first when wearing shoes (1). As a result, the electric conduction will be blocked. In view of this, apart from the insole (21), the wearable device (2) of the present invention further includes conductive socks (25) to be used in accompany with the insole (21); Referring to FIG. 9, the conductive socks (25) are made of superfine hollow Coolmax. In production, superfine metal yarns and crab yarns are combined to offer a moisture-wicking effect and 100% antibacterial disinfection and deodorization. Meanwhile, elastic high-pressure Lycra is incorporated to enhance its elasticity and tension to totally avoid the problem of looseness, so that the socks can completely cover the user's feet. The coverage with high tension can also contribute to acceleration of blood flow and alleviation of pain and soreness in the feet. Meanwhile, the superfine metal yarn combines electrical conductivity and electrostatic elimination; thus, when the user wears the shoes (1), his/her feet can form a conductive electric cycle with the anode and cathode interfaces of conductive media (22, 23) on the surface of the insole (21) through the conductive socks (25), and the required low-frequency electro-therapeutic massage and detection of human body physiological information can be realized without any interruption.

Although the invention has been explained in relation to its preferred embodiment, it is to be understood that many other possible modifications and variations can be made without departing from the spirit and scope of the invention as hereinafter claimed.

1. A wearable device with wireless Bluetooth massage function, being a conductive sensor, comprising:
an insole, said insole partitioned into anode and cathode conductive media interfaces on its surface, and extended with anode and cathode connectors respectively on either end;
an electronic structure, which is electrically connected to the anode and cathode connectors, and which includes a motherboard and a Bluetooth chipset module to offer functions of low-frequency electro-therapeutic massage and detecting the human body physiological information.

2. The wearable device with wireless Bluetooth massage function as claimed in claim 1, wherein said wearable device further includes a mobile device, said mobile device having a built-in APP, a Bluetooth unit, and a display unit; said mobile device can pair with the Bluetooth control chipset module of the insole through the Bluetooth unit, so as to issue commands for an electro-therapeutic massage mode or a physiological signal detection mode of the insole by manipulating the APP.

3. The wearable device with wireless Bluetooth massage function as claimed in claim 1, wherein said battery of the electronic device can be button cell or any other power supply that is light, thin and small.

4. The wearable device with wireless Bluetooth massage function as claimed in claim 1, wherein said shoes include slippers, house shoes etc for indoor applications, and also leather shoes, sport shoes, leisure shoes and hiking shoes etc for outdoor applications.

5. The wearable device with wireless Bluetooth massage function as claimed in claim 2, wherein said mobile device can be smart phone or tablet computer.

6. The wearable device with wireless Bluetooth massage function as claimed in claim 2, wherein said mobile device has at least one APP, a Bluetooth unit, and a display unit.

7. The wearable device with wireless Bluetooth massage function as claimed in claim 6, wherein, in the massage mode, information shown on the screen of the display unit of the mobile device at least includes: one foot (left and right)/both feet selection schema area, massage functions selection schema area, strength adjustment schema area, duration setting schema area, and ON/OFF selection schema area.

8. The wearable device with wireless Bluetooth massage function as claimed in claim 6, wherein, in the physiological information detection mode, information shown on the screen of the display unit at least includes: a selection schema area for various physiological signals (e.g., heart beat, respiration, blood pressure, body temperature, body fat etc); when any of the physiological signal schemata is selected, the collected physiological information under this schema will be displayed in the schema area in form of various statistical diagrams (e.g., bar chart, pie chart, line chart etc), or in the data area in form of data.

9. The wearable device with wireless Bluetooth massage function as claimed in claim 1, wherein the electronic structure of said wearable device can further have a configuration of a GPS positioning chipset.

10. The wearable device with wireless Bluetooth massage function as claimed in claim 1, wherein said wearable device further includes conductive socks.
